(19) United States
(12) Patent Application Publication
(10) Pub. No.: US 2010/0190152 A1
(43) Pub. Date: Jul. 29, 2010

(54) Apparatus for Analysing A Sample and Assessing its Global Position

(75) Inventor: David James Squirrell, Wiltshire (GB)

Correspondence Address: POLSINELLI SHUGHART PC, 700 W. 47TH STREET, SUITE 1000, KANSAS CITY, MO, 64112-1802, US

(73) Assignee: ENIGMA DIAGNOSTICS LIMITED, Wiltshire (GB)

(21) Appl. No.: 12/160,938

(22) PCT Filed: Jan. 22, 2007
(86) PCT No.: PCT/GB2007/000164

§ 371 Date: Aug. 29, 2008

(30) Foreign Application Priority Data
Jan. 20, 2006 (GB) 0601181.1

(51) Int. Cl. C12Q 1/68; C12M 1/34; G01S 19/42

(52) U.S. Cl. 435/6; 435/287.2; 342/357.25

(57) ABSTRACT

An apparatus for analysing a sample and assessing its global position, the apparatus comprising a portable sample analysing device and a global positioning device.

This invention relates to an apparatus for analysing a sample and assessing its global position.

The analysis of samples, for example environmental samples, may be conducted for several reasons, for instance to test for the presence of chemicals or biological material which may be harmful to humans, animals or the environment. Such testing can allow for diagnosis of disease states either of plants or animals or of contamination or pollution, whether accidental or as a result of hostile activity such as may occur in the event of chemical or biological warfare.

Diagnosis and preferably rapid diagnosis, which may be carried out on site, allows for appropriate containment or treatment measures to be implemented. This can be very important for instance in the control of infections such as foot and mouth disease or other highly contagious organisms, and for the removal or control of environmental contaminants and the like.

Sample analysis devices that can be mobilised to a site of interest are being developed with the aim of shortening diagnosis time. Examples include devices for processing fluid samples such as the PCR machine described in WO 2005/019836, which is incorporated herein by reference in its entirety. Other examples include mobile mass spectrometers, soil, air and water analysing kits, and other mobile biological and chemical analysing devices.

These portable devices are very useful in allowing rapid diagnosis or analysis of samples to be carried out on site. However, for effective treatment or containment exercises, including where necessary, the evacuation of the local population or livestock, it is important to understand the precise location of any potential hazard. This may be difficult, particularly if for example, the landscape in which the hazard is located lacks clear visible markers. Precise identification in for example, rural areas, forests, deserts or remote mountainous regions may be difficult to assess.

According to the present invention there is provided an apparatus for analysing a sample and assessing its global position, the apparatus comprising a portable sample analysing device and a global positioning device.

By combining a global positioning device with a portable sample analysing device, users are able to precisely identify the location of any hazard they have discovered as a result of the analysis. In addition the global positioning device may provide time and/or date information. Such location, time and date information may be stored if desired for each sample analysed by the sample analysing device. Thus it becomes possible to improve the logistics of any control, decontamination or evacuation exercise. By identifying the global position from which the sample has been taken, it is possible to for example, accurately set up perimeters at a safe distance from contaminated or infected sites.

A particular example of where this would be useful is for defence purposes where biological or chemical hazards are found. Having such information would allow safe perimeters to be set up around dangerous sites.

Another example of where this would be useful is in the testing of animal sets for disease, for example badger sets for TB. Using such a device will allow information to be gathered on the locations of infected sets and non-infected sets, therefore reducing the culling of non-infected animals.

As used herein the term “global positioning device” shall be taken to mean any device capable of determining its global position anywhere on earth and/or in earth's orbit. Suitable devices may for example use radio satellite navigation systems or terrestrial longwave radio systems. Examples of global positioning devices which are included within this term are GPS receivers, LORAN receivers, Omega receivers and Transit receivers. Further such devices may be available for example by way of the GLONASS receivers and Galileo receivers.

In a preferred embodiment the global positioning device is a GPS receiver such as a Navman™ chip.

As used herein the term “portable” shall be taken to mean that the apparatus can be relocated to a desired test site by hand and/or by vehicle. In an embodiment the apparatus is adapted for use in or on the land, in or on water and/or in the air. In a preferred embodiment the device is adapted for use on land.

In a particular embodiment the portable sample analysing device is for multiple use and is not therefore disposable. However, it is envisaged that it may have one or more removable or disposable chambers, and may utilise disposable analytic components such as reaction vessels, platforms, plates, strips, membranes etc. For instance, several chambers of identical dimensions can be manufactured and filled with reagents for carrying out multiple analyses. This would advantageously allow a user to select and insert a required pre-loaded chamber into the device thereby increasing the usefulness of the apparatus, and the ease of operation.

Ideally the sample analysing device is suitable for analysing the chemical, biological, optical, electrical, thermal, mechanical and/or acoustical characters of a sample to be analysed.

In a particular embodiment, the sample analysing device is a device capable of carrying out a PCR amplification of an analyte by a nucleic acid amplification reaction. Thus it suitably comprises for example, a thermal cycler and means for detecting amplification. For example, the device may further include an optical system for reading out for instance, fluorescent signals, generated by reagents such as fluorescently labelled primers and probes which are added to the reaction mixture and which generate a fluorescent signal as the reaction progresses. Such systems are known for example as the Taqman™ system, but many others such as the ResonSense™ system are available. The optical system incorporated into the device is suitably adapted to measure the signals generated within the particular assay system being used.

Suitable thermal cyclers include heating blocks, air-cooled devices but in a particular embodiment utilise an electrically conducting polymer as described for example in WO98/24548.

Additionally or alternatively the sample analysing device may be an immunoassay device, DNA sequencing device, mass spectrometers, soil, air or water analysing device other any other suitable biological or chemical analysis device.

In a particular embodiment the sample analysing device is also suitable for processing samples before analysis, for example fluid samples comprising a biological analyte.

In a particular embodiment the apparatus may display the GPS data itself. Alternatively or additionally the apparatus may further comprise a computer, for example a laptop computer or personal digital assistant (PDA or palmtop) to allow data from the analyser and/or global positioning device to be viewed, stored and/or manipulated as required. The personal digital assistant, computer or laptop computer preferably allows data to be synchronized with or sent to a remote computer.

Suitably the apparatus further comprises or is linked to a control means such that its operation may be fully automated. This advantageously reduces user error, sample contamination and user risk.

In particular the global positioning device is integral with the sample analysing device. For instance the mechanism of the global positioning device can be incorporated into the casing of the sample analysing device. In a particular embodiment, any display from the global positioning device is integrated so as to appear as an element in the sample analysing device. The precise arrangement would depend upon the precise nature of the portable sample analysing device.

In an embodiment an external arial may be used, for example in an embodiment where a case of the device is formed from steel. The external aerial may be a plug in aerial and therefore may be formed separately from the case. In an alternate embodiment an internal aerial may be used, for example in an embodiment where a case of the device is formed from a plastics material.

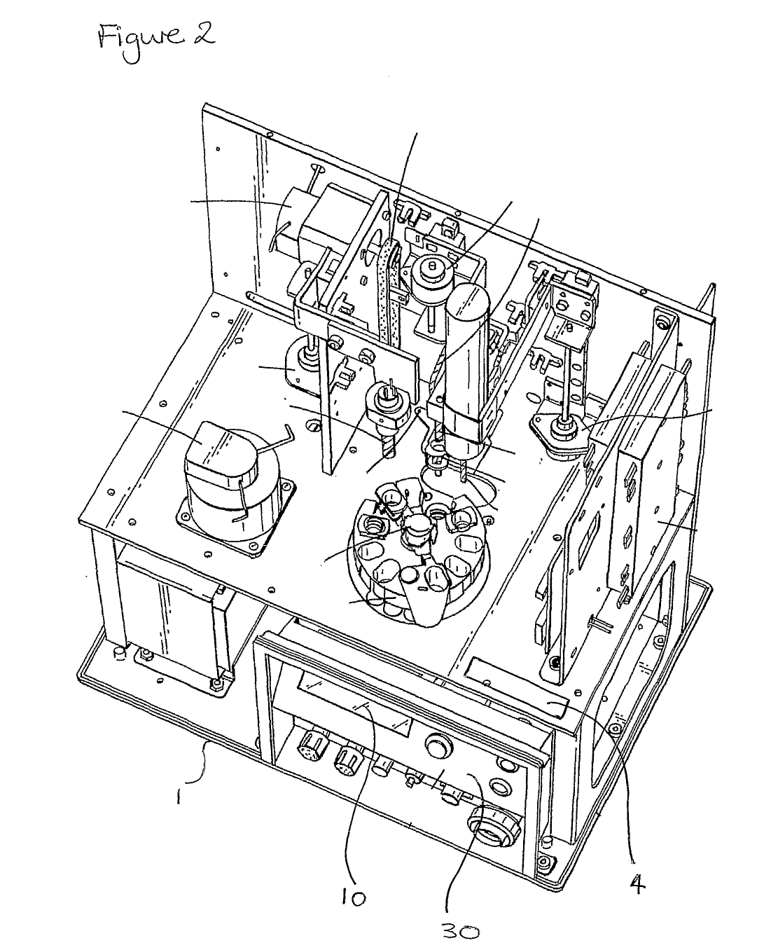

For example, in the device of WO2005/019836, the global positioning device is suitably located within the housing (1) (FIG. 2) and arranged so that the position of the device, and thus the sample being analysed is displayed upon the screen (10) located on the control panel 30.

According to a second aspect of the invention there is provided the use of an apparatus according to the present invention for the analysis of a sample and detection of its global position.

Ideally the apparatus is positioned directly on top of the site from which the sample to be analysed was taken. This will advantageously give an accurate global position reading of the sample site. If this is not possible then the apparatus may be placed at a known distance and direction from the sample site. The know distance and direction can then be factored into the data generated by the global positioning device to give an accurate indication of the global position of the sample site.

As used herein the term “accurate” when used in relation to global position shall be taken to mean accurate to within the capable limits of the global positioning device used.

The invention will now be particularly described by way of example with reference to the accompanying diagrammatic drawings in which:

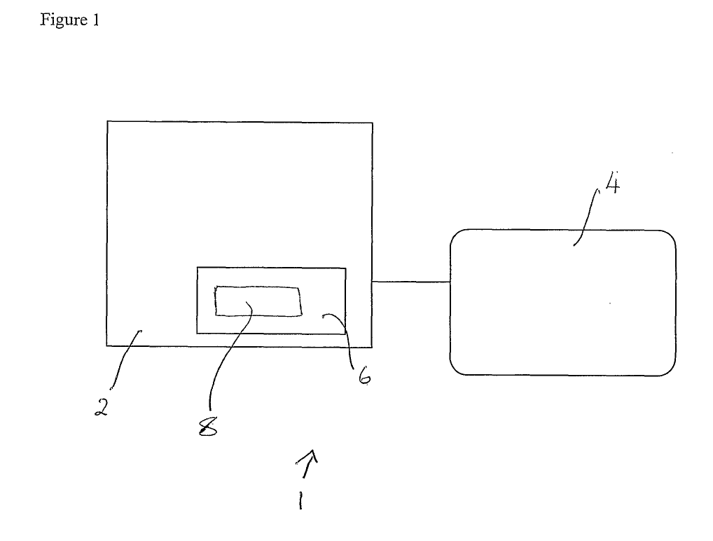

FIG. 1 shows a schematic drawing showing an embodiment of the present invention; and

FIG. 2 illustrates a particular portable sample analysing device which can be adapted to include a global positioning device.

The apparatus indicated generally at 1, comprises a portable sample analysing device 2 and a global positioning device 4.

The apparatus 1 is preferably light weight, of a simple design, requiring minimal power sources, preferably portable power sources such as batteries and the like. It may be ideally designed to work in extremes of temperature. It is preferred that the apparatus 1 is designed to operate effectively in a temperature range of from about −30° C. to about +50° C.

It is also preferred that the operation of the apparatus 1 is fully automated. This has several advantages including ease of use and reduced user error.

In order to automate the apparatus 1 it may be pre-programmed, for example by interfacing it with a computer or other suitable control means and downloading or transferring the desired programs onto the apparatus, for example assay routine programs.

The apparatus may suitably be re-programmed by interfacing it with a computer or other suitable control means and downloading or transferring the desired programs.

Data from the apparatus may be transferred from the apparatus after use, for example by downloading the data onto an interfaced computer, laptop computer or PDA.

Alternatively the apparatus may be permanently interfaced with a computer or other suitable control means 6 that has been suitably programmed, for example programmed with one or more assay routines.

Optionally the control means 6 can be chosen such that it can be programmed with more than one protocol and the user can chose the desired protocol from a suitable optional interface 8.

FIG. 2 shows a particular arrangement in which a global positioning device (4) is arranged within the housing (1) of a portable PCR machine. The electronics are arranged so that the position at any time is displayed upon screen (10).

However, many other arrangements or positions would be possible as would be appreciated by the skilled reader.

1. An apparatus for analysing a sample and assessing its global position, the apparatus comprising a portable sample analysing device and a global positioning device.

2. An apparatus according to claim 1 wherein the global positioning device is a global positioning satellite (GPS) receiver.

3. An apparatus according to claim 1 wherein the sample analysing device is for multiple use.

4. An apparatus according to claim 1 wherein the sample analysing device has one or more removable chambers.

5. An apparatus according to claim 1 wherein the sample analysing device is suitable for analysing the chemical, biological, optical, electrical, thermal, mechanical, acoustical characteristics, or combinations thereof of the sample to be analysed.

6. An apparatus according to claim 1 wherein the sample analysing device is a polymerase chain reaction (PCR) machine.

7. An apparatus according to claim 1 wherein the sample analysing device is an immunoassay device.

8. An apparatus according to claim 1 wherein the sample analysing device is a deoxyribonucleic acid (DNA) sequencing device.

9. An apparatus according to claim 1 wherein the sample analysing device is also suitable for processing samples before analysis.

10. An apparatus according to claim 1 further comprising a personal digital assistant.

11. An apparatus according to claim 1 which further comprises or is linked to a control means.

12. An apparatus according to claim 11 which is fully automated.

13. A method of analysing a sample and detecting its global position, comprising;
providing an apparatus for analysing the sample and detecting its global position, the apparatus comprising a portable sample analysing device and a global positioning device;
analysing the sample with the apparatus; and
detecting the global position of the sample with the apparatus.

14. The method according to claim 13 further comprising positioning the apparatus directly on top of a sample site.

15. The method according to claim 13 further comprising placing the apparatus at a known distance and direction from a sample site and factoring the known distance and direction into a reading generated by the global positioning device to give an accurate indication of the global position of the sample site.
